(19) United States
(12) Patent Application Publication
(10) Pub. No.: US 2010/0034778 A1
(43) Pub. Date: Feb. 11, 2010

(54) STEM CELLS

(75) Inventors: Donald P. McDonnell, Durham, NC (US); John P. Chute, Durham, NC (US)

Correspondence Address: NIXON & VANDERHYE, PC, 901 NORTH GLEBE ROAD, 11TH FLOOR, ARLINGTON, VA, 22203, US

(73) Assignee: DUKE UNIVERSITY, Durham, NC (US)

(21) Appl. No.: 12/066,957

(22) PCT Filed: Sep. 14, 2006
(86) PCT No.: PCT/US2006/035863

§ 371 Date: Feb. 5, 2009

Related U.S. Application Data
(60) Provisional application No. 60/716,500, filed on Sep. 14, 2005.

(51) Int. Cl. A61K 35/12; C12N 5/02; C12N 5/071; C12Q 1/02

(52) U.S. Cl. 424/93.7; 435/375; 435/366; 435/372; 435/29

(57) ABSTRACT

The present invention relates, in general, to stem cells and, in particular, to a method of expanding human stem cells using a retinoic acid receptor modulator.

This application claims priority from U.S. Provisional Application No. 60/716,500, filed Sep. 14, 2005, the entire content of which is incorporated herein by reference.

### TECHNICAL FIELD

The present invention relates, in general, to stem cells and, in particular, to a method of expanding stem cells using a retinoic acid receptor modulator.

### BACKGROUND

Hematopoietic stem cells (HSCs) possess the unique capacity to self-renew and give rise to all mature lymphophematopoietic progeny throughout the lifetime of an individual (Osawa et al, Science 273:242-245 (1996), Sorrentino, Nat. Rev. Immunol. 4:878-888 (2004)). Several molecular pathways that regulate HSC self-renewal have now been identified, including Notch (Varnum-Finney et al, Nat. Med. 6:1278-1281 (2000)), HOXB4 (Krosl et al, Nat. Med. 9:1428-1432 (2003)), Wnt (Reya et al, Nature 423:409-414 (2003)) and bone morphogenetic protein signaling pathways (Bhardwaj et at, Nat. Immunol. 2:172-180 (2001)). The osteoblastic niche for HSCs within the bone marrow (BM) has also been characterized (Calvi et al, Nature 425:841-846 (2003), Zhang et al, Nature 425:836-841 (2003)). Despite these advances in understanding HSC biology, clinical methods to amplify human HSCs have yet to be realized and characterization of the pathways that regulate HSC self-renewal continues to evolve.

The biological actions of retinoids are mediated by the retinoic acid receptors, RAR and RXR, ligand-dependent transcription factors that are expressed in the nuclei of target cells (Chambon, FASEB J. 10:940-954 (1996), Collins, Leukemia 16:1896-1905 (2002), Zechel, Mol. Endocrinol. 19:1629-1645 (2005)). Through its actions on these receptors, all-trans retinoic acid (ATRA), a derivative of Vitamin A, induces cellular differentiation, tissue patterning and embryonic development in vertebrates (Chambon, FASEB J. 10:940-954 (1996), Collins, Leukemia 16:1896-1905 (2002), Zechel, Mol. Endocrinol. 19:1629-1645 (2005), Zile, J. Nutr. 131:705-708 (2001)). Treatment of myeloid progenitors with ATRA induces terminal granulocytic differentiation (Collins, Leukemia 16:1896-1905 (2002), Tocci et al, Blood 88:2878-2888 (1996)) and ATRA is used therapeutically to induce the differentiation of acute promyelocytic leukemia cells, in which the characteristic 15; 17 translocation results in a fusion protein (PML-RARα) which harbors dominant negative activity against the RARα receptor (Tallman et al, N. Engl. J. Med. 337:1021-1028 (1997)). It has also been observed that the fold expansion of human cord-blood (CB)-andbone marrow (BM) nonobese diabetic/severe combined immunodeficiency (NOD/SCID) mouse repopulating cells (SRCs) in co-culture with vascular endothelial cells correlates linearly with the amplification of CD34⁺ cells lacking expression of CD38, which is dependent on RARA signaling (Mehta et al, Blood 89:3607-3614 (1997), Chute et al, Blood 100:4433-4439 (2002), Chute et al, Blood 105:576-583 (2005)).

The present invention results, at least in part, from functional screening studies of RAR and RXR modulators for their ability to inhibit HSC differentiation in vitro. These studies have resulted in the identification of LGD1506 as an agent effective at inducing HSC expansion. This compound is a Selective RXR Modulator, capable of mimicking RXR actions in some contexts while functioning as an antagonist in others.

### SUMMARY OF THE INVENTION

The invention relates generally to stem cells. More specifically, the invention relates to methods of expanding stem cells using retinoic acid receptor modulators.

Objects and advantages of the present invention will be clear from the description that follows.

### BRIEF DESCRIPTION OF THE DRAWINGS

FIGS. 1A and 1B. Treatment with ATRA accelerates the differentiation of primary human HSCs. FACS-sorted CB CD34⁺CD38⁻lin⁻ cells were cultured with thrombopoietin, stem cell factor (SCF) and F1+3 ligand (TSF) alone versus TSF+ATRA×7 days. (FIG. 1A) CD34 and CD38 surface expression is shown on day 0 cells (top), their progeny following TSF culture (middle) and their progeny following TSF+ATRA×7 days (bottom). ATRA induced a marked loss of CD34⁺ cells and CD34⁺CD38− cells in culture as compared to input or TSF culture, consistent with differentiation during culture. (FIG. 1B) The progeny of TSF+ATRA cultures also contained signficantly less CFCs as compared to TSF cultured progeny, suggesting that ATRA induced the terminal differentiation or apoptosis of stem and progenitor cells in culture.

FIGS. 2A-2E. Modulation of RXR inhibits HSC differentiation and promotes HSC expansion. (FIG. 2A) The frequency of CD34⁺CD38⁻ cells was measured following culture of BM CD34⁺CD38⁻lin⁻ with TSF plus either ATRA, LGD0815, LGD0268 or LGD1506. Addition of LGD1506 to TSF differentially maintained CD34⁺CD38⁻ cells in culture compared to the other retinoid and rexinoid ligands. LGD1506 supported the maintenance of both BM (FIG. 2B) and CB CD34⁺CD38⁻cells (FIG. 2C) in culture. (FIG. 2D) LGD506+TSF culture induced a 4-fold increase in CD34⁺CD38⁻ cell numbers compared to input (left) and caused a 50% reduction in CFC production during culture as compared to TSF alone (right). (FIG. 2E) Neither 5×10³ day 0 BM CD34⁺CD38⁻lin⁻ cells nor their progeny following TSF culture demonstrated long-term engraftment in any NOD/SCID mice, but the progeny of the identical dose of BM CD34⁺CD38⁻ lin⁻ cells following culture with LGD1506+TSF or demonstrated human repopulation in 50-80% of transplanted mice.

FIGS. 3A and 3B. Treatment with LGD1506 maintains HOXB4 expression in HSCs. RNA was isolated from multiple replicates of FACS-sorted CB CD34⁺CD38⁻lin⁻ cells at day 0 and their progeny following culture with TSF alone or LGD1506+TSF. The RNA was reverse transcribed and the expression of HOXB4 and Notch 1 was analyzed by quantitative real-time PCR. (FIG. 3A) The expression of HOXB4 in CD34⁺CD38⁻ cells was significantly reduced following short term culture with TSF, whereas treatment with LGD1506 prevented the downregulation of HOXB4 expression over time. (FIG. 3B) The expression of Notch was also significantly reduced following TSF culture but LGD1506 treatment altered this decline in Notch expression over time.

FIG. 4. Structure of LGD1506.

### DETAILED DESCRIPTION OF THE INVENTION

The present invention relates to a method of promoting expansion of stem cells, or progenitor cells, while inhibiting differentiation of such cells. The method comprising contacting the cells, for example, human hematopoietic stem cells (HSCs), with a modulator of the retinoic acid receptor RXR under conditions such that expansion is effected.

Stem cells suitable for expansion in accordance with the invention include, for example, HSCs, neuronal stem cells and muscle stem cells. HSCs suitable for expansion can be obtained, for example, from bone marrow, umbilical cord blood or peripheral blood. Stem cells suitable for use can be separated from mixed populations of cells and cultured in the presence of an RXR modulator. The thus cultured cells can then be harvested.

Stem cells can be distinguished from most other cells by the presence or absence of particular antigenic marker antigens, such as CD34, that are present on the surface of these cells and/or by morphological characteristics. One phenotype of a highly enriched human stem cell fraction has been reported as CD34⁺, Thy-1⁺ and Lin⁻, however, the present invention is not limited to the expansion of this stem cell population.

A CD34⁺ enriched human stem cell fraction can be separated by a number of art-recognized techniques, including affinity columns or beads, magnetic beads or flow cytometry using antibodies directed to surface antigenics such as CD34⁺. The CD34⁺ progenitors can be divided into subpopulations characterized by the presence or absence of coexpression of different lineage-associated cell surface associated molecules. Immature progenitor cells do not express lineage associated markers such as CD38.

The separated cells can be incubated in a selected culture medium, for example, in a culture dish or flask, a sterile bag or hollow fiber. Various hematopoietic growth factors can be utilized in order to selectively expand cells. Representative factors include thrombopoietin, SCF and flt-3 ligand, or combinations thereof. Proliferation of the stem cells can be monitored by counting the number of stem cells using standard techniques (e.g., hemacytometer) or by flow cytometry prior and subsequent to incubations.

Retinoic acid receptor modulators suitable for use in accordance of the invention include selective RXR modulators, such as LGD1506 (FIG. 4). The invention includes methods of identifying RXR modulators appropriate for use in effecting stem cell expansion. Candidate compounds can be screened for their ability to bind to RXR (e.g., specifically), for their ability to block cytokine-induced differentiation of stem cells (e.g., HSCs) and/or for their ability to block stem cell differentiation by modulating HOXB4 expression/activity. The invention does not include the use of AGN 194310 (Prus et al, Leuk. Lymph. 45:1025 (2004)) in expanding stem cells or subpopulations thereof.

The RXR modulators of the invention, advantageously used in combination with TSF (or other appropriate cytokine combination), result in the amplification of pluripotent cells that maintain normal differentiation capacity.

Stem cells expanded ex-vivo using an RXR modulator of the invention can be used in the treatment of various diseases, including those characterized by decreased levels of either myeloid, erythroid, lymphoid or megakaryocyte cells of the hematopoietic system. In addition, they can be used to cultivate mature myeloid and/or lymphoid cells. Among conditions susceptible to treatment with hematopoietic cells expanded in accordance with the invention is leucopenia induced, for example, by exposure to viruses or radiation, or as a side effect of cancer therapy. The expanded cells of the invention can also be useful in preventing or treating bone marrow suppression or hematopoietic deficiencies that occur in patients treated with a variety of drugs.

The dosage regimen involved in ex vivo expansion of cells and methods for treating the above-described conditions can be determined by one skilled in the art and can vary with the RXR modulator, the patient and the effect sought.

In addition to the ex vivo expansion of stem cells for therapeutic purposes (i.e., cord blood transplantation) the RXR modulators can be used as systemic therapeutics, for example, for treating patients undergoing chemotherapy and/or radiotherapy to accelerate their hematopoietic recovery, as well as other patients suffering from blood cell disorders/deficiencies, including anemias (e.g., sickle cell anemia).

Certain aspects of the invention can be described in greater detail in the non-limiting Example that follows.

### Example

### Experimental Details

Isolation of Human BM and CB CD34⁺CD38⁻lin− cells

Whole BM and CB units were obtained from the Duke University Stem Cell Laboratory within 48 hours of collection. Volume reduction of CB units was accomplished by 10 minute incubation at room temperature with 1% Hetastarch (Abbott Laboratories, North Chicago, Ill.), followed by centrifugation at 700 rpm for 10 minutes to facilitate component separation. The buffy coat was collected and washed twice with Dulbecco's phosphate buffered saline (PBS; Invitrogen, Carlsbad, Calif.) containing 10% heat-inactivated fetal bovine serum (FBS; Hyclone, Logan, Utah), 100 U/ml penicillin, and 10 ug/ml streptomycin (1% pcn/strp; Invitrogen). Cell pellets were resuspended in PBS with 10% FBS and 1% pcn/strp and overlaid onto Lymphoprep (Axis-Shield, Olso, Norway) and centrifuged at 1500 rpm for 30 minutes to isolate the mononuclear cell (MNC) fraction. MNCs were collected and washed twice before proceeding to lineage depletion.

Lineage depletion was conducted using the Human Progenitor Enrichment Cocktail (Stem Cell Technologies, Vancouver, Canada) which contains monoclonal antibodies (mAbs) to human CD2, CD3, CD14, CD16, CD9, CD56, CD66b, and Glycophorin A, according to the manufacturer's suggested protocol. Briefly, CB or BM MNCs were resuspended at 5−8×10⁷ cells/ml in PBS with 10% FBS and 1% pcn/strp and incubated with 100 μl/ml antibody cocktail for 30 minutes followed by incubation with 60 μl/ml magnetic colloid for 30 minutes. Cells were then magnetically depleted on a pump fed negative selection column (Stem Cell Technologies) using the manufacturer's recommended procedure. Lin-cells were washed twice, quantified by hemacytometer count and cryopreserved in 90% FBS and 10% dimethylsulfoxide (DMSO; Sigma-Aldrich, St. Louis, Mo.) or utilized directly for further experiments.

Lin⁻ CB or BM cells were thawed, washed once in Iscove's Modified Dulbecco's Medium (IMDM; Invitrogen) containing 10% FBS and 1% pcn/strp, counted, and resuspended at 5−10×10⁷/ml. Immunofluorescent staining was conducted using anti-human CD34-fluorescein isothiocyanate (FITC) and anti-human CD38-phycoerythrin (PE) monoclonal antibodies (Becton Dickinson, San Jose, Calif.), for 30 minutes on ice. Stained cells were washed twice and resuspended at 1×10⁷ cells/ml in IMDM with 10% FBS and 1% pcn/strp. Analysis and sterile cell sorting was conducted using a FACSvantage flow cytometer (Becton Dickinson) to isolate CD34⁺CD38⁻ and CD34⁺CD38⁺ subsets. The CD34⁺CD38⁻ sort gate was set to collect only those events falling in the lowest 5% of PE fluorescence within the total CD34⁺ population, to ensure acquisition of highly purified CD34⁺CD38⁻ cells.

Analysis of In Vitro Hematopoietic Activity of Human CD34⁺CD38⁻lin⁻ Cells Following Culture with LGD1506

Primary human BM and CB CD34⁺CD38⁻lin cells were placed in culture with 20 ng/mL thrombopoietin, 100 ng/mL stem cell factor (SCF), and flt-3 ligand (TSF), a cytokine combination previously found to induce human stem and progenitor cell proliferation and differentiation in vitro (Chute et al, Blood 105:576-583 (2005), Chute et al, Stem ells 22:202-215 (2004)).

In order to assess the hematopoietic effects of retinoid agonists on purified human HSCs, BM and CB CD34⁺CD38⁻lin⁻ cells were placed in culture with TSP with and without 1 μM all-trans retinoic acid (ATRA), an agonist of RAR. Total cell expansion, immunophenotype, colony forming cell (CFC) content and morphologic analysis was performed on ATRA-treated progeny and compared with both day 0 CD34⁺CD38⁻lin⁻ populations and the progeny of TSF alone to assess the impact of retinoid agonism on the differentiation of human HSCs. This analysis was extended further by cultivating BM and CB CD34⁺CD38⁻lin⁻ cells with TSF coupled with ligands with specific affinity for either RAR or RXR (provided courtesy of Ligand Pharmaceuticals, Inc., San Diego, Calif.). The hematopoietic activities of LGD0815, an RARA agonist, LGD0268, an RXR agonist, and LGD1506, an RXR selective modulator, in combination with TSF, were evaluated via measurement of total cell expansion, immunophenotype analysis, CFC content and morphologic analysis of cultured progeny versus day 0 (input) BM or CB CD34⁺CD38⁻lin⁻ cells.

### In Vivo Long-Term Repopulating Assays in NOD/SCID Mice

NOD/SCID mice (Schulz et al, J. Immunol. 154:180-191 (1995)) were transplanted with either day 0 FACS-sorted BM or CB CD34⁺CD38⁻ lin⁻ HSC-enriched cells or the progeny of CD34⁺CD38⁻lin− cells cultured with TSF alone or TSF supplemented with 1 μM LGD506, an RXR-specific ligand. Cells were transplanted via tail vein injection after irradiating NOD/SCID mice with 300 cGy using an X-ray irradiator as previously described (Chute et al, Blood 100:4433-4439 (2002), Chute et al, Blood 105:576-583 (2005)). Day 0 CD34⁺CD38⁻lin− HSCs and their progeny were co-transplanted with 2×10⁴ CD34⁺CD38⁻lin⁻ accessory cells to facilitate engraftment as previously described (Bonnet et al, Bone Marrow Transpl. 23:203-209 (1999), Bhatia et al, Proc. Natl. Acad. Sci. USA 94:5320-5325 (1997)).

All mice in each group were sacrificed at week 8 and marrow samples were obtained by flushing their femurs with IMDM at 4° C. Red cells were lysed using red cell lysis buffer (Sigma-Aldrich) and flow cytometric analysis of human hematopoietic engraftment was performed as previously described using commercially available mAbs against human leukocyte differentiation antigens to identify engrafted human leukocytes and discriminate their hematopoietic lineages (Chute et al, Blood 100:4433-4439 (2002), Chute et al, Blood 105:576-583 (2005), Trischmann et al, J. Hematother. 2:305-313 (1993)).

### Statistical Analysis and SRC Frequency Measurements

Comparisons of data from in vitro experiments were made using the Student's t test. For purposes of the limiting dilution analysis, a transplanted mouse was scored as positively engrafted if ≧0.1% of the marrow cells expressed human-CD45 via high resolution FACS analysis. This criteria is consistent with previously published criteria for human cell repopulation in NOD/SCID mice (Chute et al, Blood 100:4433-4439 (2002), Dorrell et al, Blood 95:102-110 (2000)). SRC frequency in each cell source was calculated using the maximum likelihood estimator as described previously by Taswell (J. Immunol. 126:1614-1619 (1981), Wang et al, Blood 89:3919-3924 (1997), Ueda et al, J. Clin. Invest. 105:1013-1021 (2000)). Confidence intervals for the frequencies were calculated using the profile likelihood method, and the likelihood ratio test was used to confirm the fit of the model.

### Real Time PCR Analysis of LGD506-Treated HSCs

Extraction of total RNA from Day 0 and Day 7 CB CD34⁺ populations was done using a RNeasy Mini spin column (Qiagen, Valencia, Calif.) according to the manufacturer's recommended protocol. Total RNA isolation from Day 0 CB CD34⁺CD38⁻lin⁻ and the resultant Day 7 progeny was conducted on 1×10⁴ cells/sample, using the RNAqueous-Micro kit (Ambion, Austin, Tex.), using the manufacturer's suggested protocol. Briefly, total RNA was isolated from CD34⁺CD38⁻lin⁻ cells according to the manufacturer's instructions for the RNaqueous®—Micro (Ambion) and reversed-transcribed to cDNA using iScript™ cDNA synthesis Kit (Biorad, Hercules, Calif.). cDNA concentrations were measured with a fluorometer (Turner Designs, Sunnyvale, Calif.) using RiboGreen reagent (Invitrogen). PCR amplification reactions were performed in 13 μl and contained equal amounts of cDNAs, 6.5 μL of iQ SYBR green supermix (Bio-Rad), 0.2 μM of each forward and reverse gene-specific primers for genes of interest and the normalization gene 36B4. PCR was performed on an iCycler (Bio-Rad) according to the following cycling conditions: an initial cycle of 15 min at 95° C.; 45 cycles of 45 sec at 95° C., 15 sec at 55° C., and 15 sec at 72° C.; followed by a melt-curve analysis cycle with steps of 10 sec each at 0.5° C. increments from 60 to 95° C. Amplification rates were visualized and analyzed on ICYCLER IQ optical system software version 3.0 (Bio-Rad). Gene-specific primers were purchased from Integrated DNA Technologies (Coralville, Iowa).

### Results

Treatment with All-Trans Retinoic Acid (ATRA) Promotes the Differentiation of Human HSCs

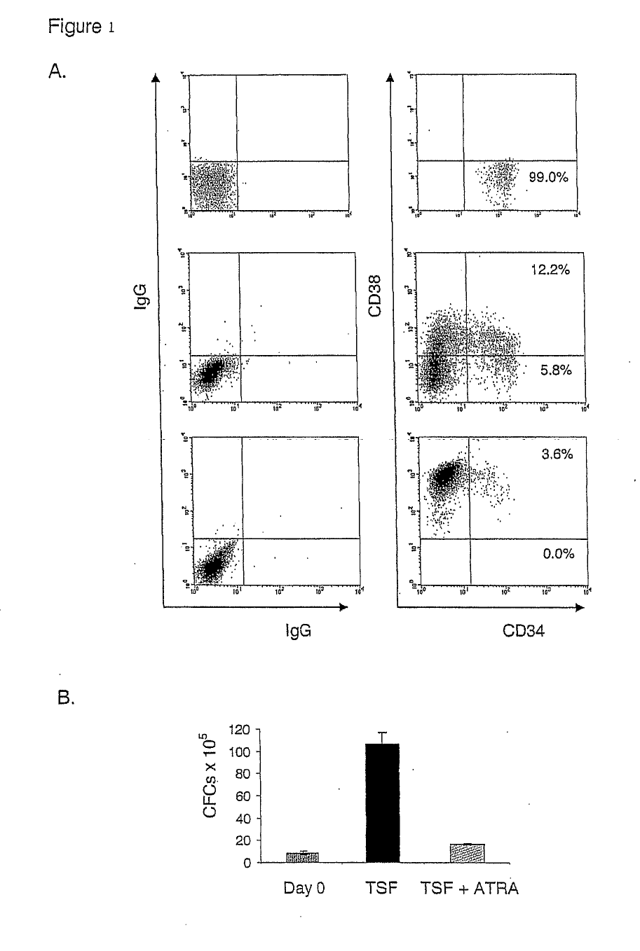

The effects of ATRA on primary CB CD34⁺CD38⁻lin⁻ cells in culture were evaluated. When CB CD34+CD38−Iin− cells were cultured with TSF+1 μM ATRA, the percentage of CD34⁺ cells declined relative to TSF alone, and no CD34⁺CD38⁻ cells remained in culture at day 7 (FIG. 1A). The number of CFCs were also decreased in the ATRA+TSF treated cultures compared to cultures with TSF alone, suggesting that ATRA either inhibited CFC formation or promoted terminal differentiation, thereby reducing CFC numbers (FIG. 1B). Taken together, these analyses suggested that ATRA enhanced the differentiation of HSCs during culture with TSF.

### Selective Modulation of RXR Inhibits Human HSC Differentiation and Induces the Expansion of SRCs

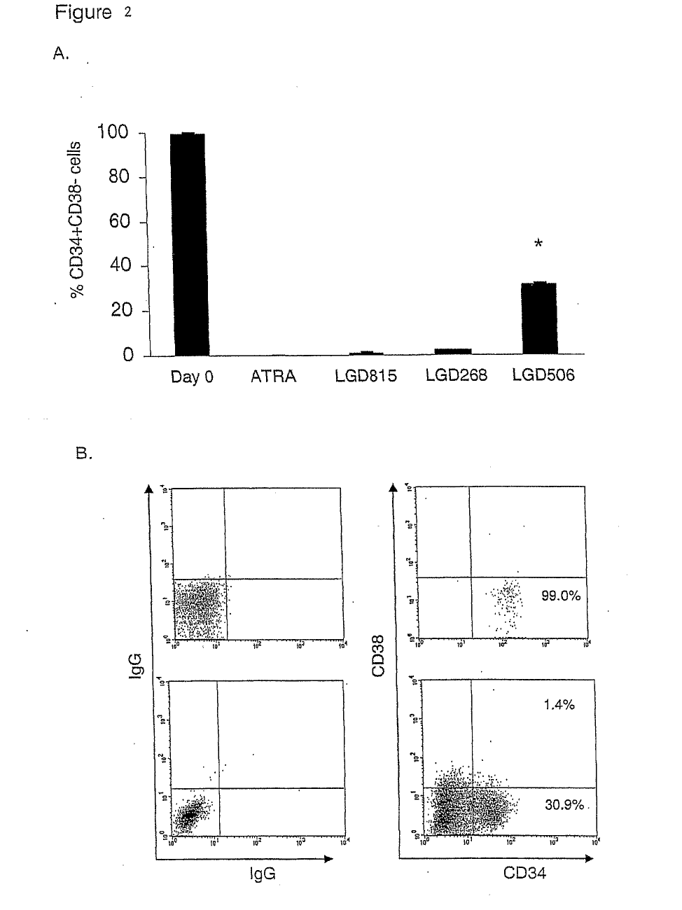

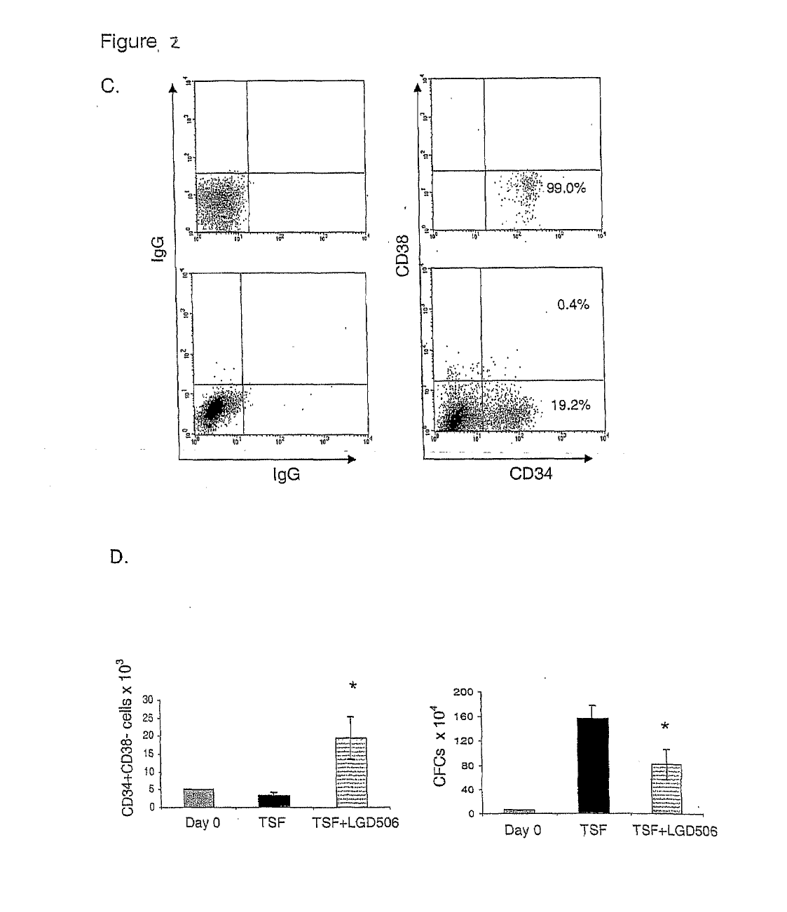

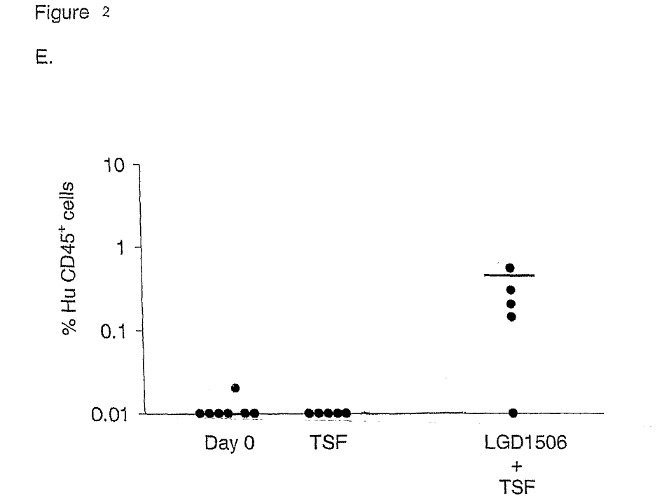

Although ATRA is a high affinity agonist of RAR, it is also capable of spontaneously isomerizing to 9-cis retinoic acid, a specific agonist of RXR (Levin et al, Nature 355:359-361 (1992)). To assess the relative contribution of each of these receptor subtypes, the effects of several RAR and RXR modulators on HSC growth were evaluated and compared to those of ATRA. Treatment of BM CD34⁺CD38⁻lin⁻ cells with either TSF alone, TSF+ATRA, or TSF+1 μM LGD0815, an RARA antagonist, or TSF+1 μM LGD0268, an RXR agonist, caused a decline in the percentage of CD34⁺CD38⁻ cells by day 7. Conversely, following culture with TSF+1 μM of the RXR modulator, LGD1506, 31% of the day 7 population remained CD34⁺CD38⁻ (FIGS. 2A, B). The addition of LGD1506 produced a similar maintenance of CD34⁺CD38⁻ cells in CB cultures (FIG. 2C). Coupled with a 13-fold total cell expansion, LGD1506+TSF supported a 4-fold increase in BM CD34⁺CD38⁻ cells compared to day 0, whereas TSF alone caused a decline in CD34⁺CD38⁻ cell numbers over time (FIG. 2D). The progeny of LGD1506+TSF cultures also contained 2-fold less CFCs compared to TSF-cultured cells, indicating an inhibition of HSC differentiation during culture. Morphologic examination of the progeny of LGD1506+TSF cultures also revealed a predominance of immature blasts, suggesting that LGD1506 treatment maintained more primitive hematopoietic cells in culture (data not shown). Taken together, these data suggested that the RXR modulator, LGD1506, inhibited HSC differentiation during culture.

In order to determine whether treatment with LGD1506 promoted the expansion of BM HSCs, NOD/SCID repopulating cell assays were performed. Over a dose range of 1−2.5×10³ cells, no huCD45+ cell repopulation was detected in mice transplanted with either day 0 BM CD34⁺CD38⁻lin⁻ cells or their progeny following culture with TSF alone or, TSF+LGD1506 (i=35 mice). At a dose of 5×10³, neither mice transplanted with day 0 BM CD34⁺CD38⁻lin⁻ cells (0 of 5) nor their progeny following TSF culture demonstrated huCD45⁺ cell repopulation (0 of 5). In contrast, 4 of 5 mice (80%) transplanted with the progeny of 5×10³ BM CD34⁺CD38⁻lin⁻ cells cultured with LGD1506+TSF showed huCD45+ cell repopulation (mean 0.2%, FIG. 2E). These results demonstrated that treatment with LGD1506 promoted the expansion of human BM SRCs. Importantly, in all mice engrafted with LGD1506-treated cells, normal multilineage differentiation was evident in vivo, confirming that LGD1506+TSF amplified pluripotent cells which maintained normal differentiation capacity. Poisson statistical analysis indicated that the SRC frequency within the LGD1506+TSE cultures was 1 in 3,100 (CI, 1/1,000−1/11,000). Since 0 of 37 mice transplanted with 1−5×10³ day 0 BM CD34⁺CD38⁻lin⁻ cells or TSF-cultured cells demonstrated huCD45⁺ cell engraftment, the SRC frequency in these groups was clearly lower than LGD1506-treated cells, but only 1-sided confidence intervals could be developed, with each having an SRC frequency of <1/12,000. Therefore, LGD1506 treatment was associated with at least a 4-fold increase in BM SRCs compared to input or their progeny following TSF culture.

### Selective Activation of RXR Upregulate HOXB4 in Human HSCs

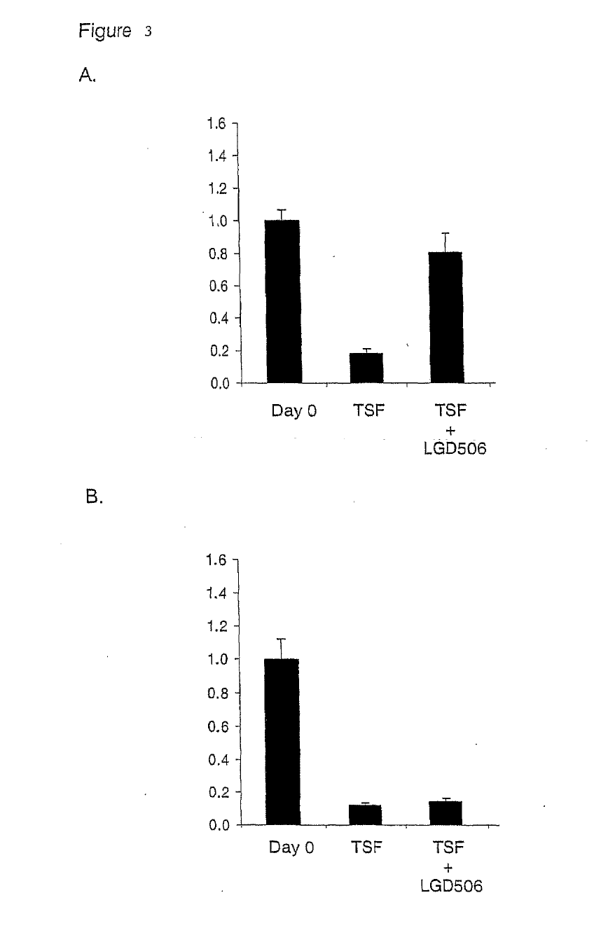

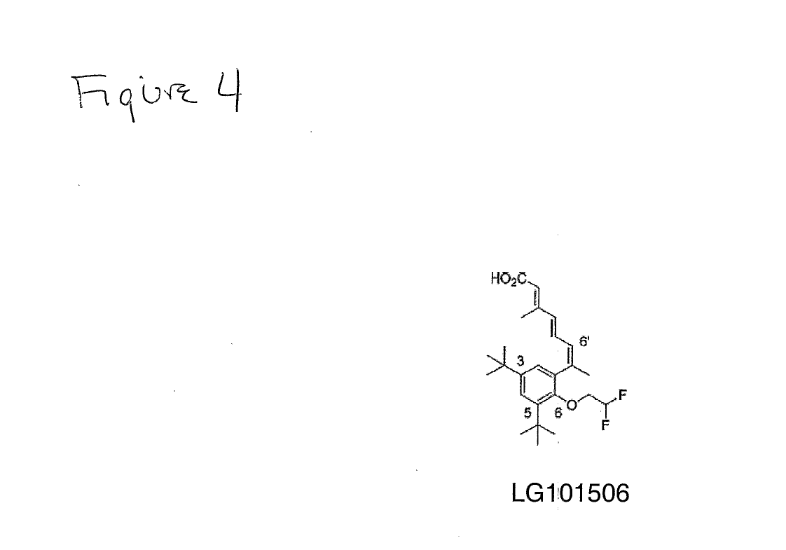

Since HOXB4 and Notch have established roles in HSC self-renewal (Varnum-Finney et al, Nat. Med. 6:1278-1281 (2000), Krosl et al, Nat. Med. 9:1428-1432 (2003)), a decision was made to determine whether RXR modulation might be regulating HSC self-renewal by altering the transcription of either of these target genes. Interestingly, culture of primary CB CD34⁺CD38⁻lin⁻ cells with TSF alone caused a 5-fold decrease in HOXB4 transcription compared to day 0 CB CD34⁺CD38⁻lin⁻ cells, whereas the addition of LGD1506 to TSF maintained HOXB4 expression at 80% and 70% of input levels, respectively (FIG. 3). Conversely, treatment with LGD1506 did not alter Notch transcription compared to TSF alone. Taken together, these data indicate that RXR modulation may promote HSC self-renewal via discrete interactions with other established pathways, such as HOXB4, although the organization of these signals is yet to be elucidated.

Summarizing, the direct effects of RAR and RXR selective ligands on HSC fate were examined. The results indicated that ATRA, which is an agonist of RAR (Levin et al, Nature 355:359-361 (1992)), induced pronounced differentiation and myelbid maturation of human HSCs by day 7. However, the RXR-selective modulator, LGD1506, inhibited HSC differentiation and induced a 4-fold expansion of both phenotypically primitive CD34⁺CD38⁻ cells and SRCs compared to either day 0 CD34⁺CD38⁻lin⁻ cells or their TSF cultured progeny. These data indicate that selective modulation of RXR with LGD1506, coupled with TSF, promotes HSC self-renewal. The contribution of RXR signaling in HSC fate has not been previously described.

It is known that RXR and RAR modulators can function as either agonists or antagonists depending on the environment in which they are studied, similar to selective estrogen receptor modulators (SERMs) (Huang et al, Mol. Endocrinol. 16:1778-1792 (2002)). Indeed, it has previously been shown in vitro that LGD1506 can function as an agonist or antagonist in different cell and promoter contexts (M. Leibowitz et al., unpublished data). The RXR is unique among the nuclear receptors in that it can function with many nuclear receptors (NRs) as an obligate partner required for both DNA binding and transcriptional activation. It has been demonstrated conclusively that the overall activity of a given NR:RXR complex can be differentially regulated by the nature of the bound rexinoid (Li et al, J. Biol. Chem. 279:7427-7437 (2004)). By exploiting this phenomenon, it has been possible to develop rexinoids that activate some heterodimeric complexes while inhibiting others. In this regard, it is important to note that LGD1506 has been shown to inhibit the RXR:LXR heterodimer while having neutral or inhibitory effects on the RXR:RAR heterodimer (M. Leibowitz et al., unpublished data). It is interesting to postulate, therefore, that its inhibitory or neutral effects on RXR:RAR and inhibitory effects on RXR:LXR, may be relevant to HSC biology. Of note, it has been found that LXR^{a} mRNA is quite highly expressed in CD34⁺CD38⁻lin⁻ cells.

In conclusion, modulation of retinoid signaling via selective modulation of RXR is sufficient to induce the expansion of human HSCs.

All documents and other information sources cited above are hereby incorporated in their entirety by reference.

What is claimed is:

1. A method of promoting expansion of stem cells or progenitor cells, said method comprising contacting said cells with a modulator of RXR under conditions such that said expansion is effected, wherein said compound is not HOXB4.

2. The method according to claim 1 wherein cells are hematopoietic stem cells, neuronal stem cells or muscle stem cells.

3. The method according to claim 2 wherein said cells are human hematopoietic stem cells (HSCs).

4. The method according to claim 3 wherein said HSCs are obtained from bone marrow, umbilical cord blood or peripheral blood.

5. The method according to claim 1 wherein said cells are CD34⁺, Thy-1⁺, Lin⁻ stem cells.

6. The method according to claim 1 further comprising contacting said cells with an amount of a hematopoietic growth factor sufficient to effect said expansion.

7. The method according to claim 6 wherein said growth factor is selected from the group consisting of thrombopoietin, ScF and flt-3 ligand.

8. The method according to claim 1 wherein said RXR modulator is LGD1506.

9. The method according to claim 8, wherein said method further comprises contacting said cells with TSF.

10. A method of identifying a compound that promotes expansion of stem cells or progenitor cells and inhibits differentiation of said cells, said method comprises contacting said compound with RXR and detecting binding of said compound to RXR, wherein a compound that binds RXR is a candidate compound for promoting expansion of said cells.

11. A method of treating a patient suffering from a disease or disorder associated with decreased levels of myeloid, erythroid, lymphoid or megakaryocytes comprising administering to said patient stem cells expanded ex vivo according to the method of claim 1 under conditions such that said treatment is effected.

12. The method according to claim 11 wherein said patient is suffering from leucopenia.

13. A method of accelerating hematopoietic recovery in a patient in need thereof comprising administering to said patient an RXR modulator in an amount sufficient to effect said acceleration.
